(19) United States
(12) Patent Application Publication
(10) Pub. No.: US 2015/0305757 A1
(43) Pub. Date: Oct. 29, 2015

(54) METHOD AND APPARATUS FOR ENCLOSURE AND REMOVAL OF CALCULI AND FOREIGN BODIES

(71) Applicant: NatEmack Holding, LLC, Atlanta, GA (US)

(72) Inventors: Emily Brayman, Atlanta, GA (US); Natalie Mitchell, Atlanta, GA (US); Jack Austin, Atlanta, GA (US)

(21) Appl. No.: 14/697,002

(22) Filed: Apr. 27, 2015

Related U.S. Application Data
(60) Provisional application No. 61/984,979, filed on Apr. 28, 2014.

(51) Int. Cl. A61B 17/221; A61B 17/22

(52) U.S. Cl. CPC A61B 17/221; A61B 17/22031; A61B 2017/22035; A61B 2017/2215

(57) ABSTRACT

The embodiments of the present disclosure may provide an apparatus used in ureteroscopy, a widespread method of treating kidney stones by enclosing the stone in a sheath before breaking it into a plurality of fragments with a fragmenting device. The configuration of the disclosed mesh top to a sheath may improve the practice of ureteroscopy by facilitating the removal of kidney stone fragments. The apparatus may comprise a sheath; a mesh top comprising: a first end having an opening into the interior of the mesh top, and a second end being attached to the sheath; and an enclosing means, wherein the mesh top is configured to: receive an object within an interior of the mesh top, enclose, via the enclosing means, the object within the mesh top, receive a fragmenting device through a second end, and retain fragments of the object caused by an operation of the fragmenting device.

### RELATED APPLICATION

Under provisions of 35 U.S.C. §119(e), the Applicants claim the benefit of U.S. provisional application No. 61/984979, filed Apr. 28, 2014 by the same inventors, which is incorporated herein by reference.

It is intended that each of the referenced applications may be applicable to the concepts and embodiments disclosed herein, even if such concepts and embodiments are disclosed in the referenced applications with different limitations and configurations and described using different examples and terminology.

### FIELD OF DISCLOSURE

The present disclosure generally relates to removal of calculi and foreign bodies.

### BACKGROUND

Kidney stones may be removed in a plurality of ways. Common methods of treatment include Extracorporeal Shock Wave Lithotripsy (ESWL), Percutaneous Nephrolithotomy (PN) and Ureteroscopy. ESWL may often lead to kidney damage, postoperative complications, and a high recurrence rate. PN is an invasive procedure that involves an incision through the skin through which a device is guided into the kidney, necessitating extended recovery time.

Compared to patients treated using ESWL or PN, ureteroscopy patients recover more quickly and suffer fewer post-operative complications. However, ureteroscopy procedures create stone fragments that may take hours to remove. Fragments are an issue with all treatment methods because fragments greater than 1 mm are clinically significant and, if left in the system, can re-aggregate and form additional stones. Consequently, up to fifty percent of all stone-formers have another stone after their first treatment.

Physicians performing ureteroscopy procedures emphasize that the duration of the procedure is the most important aspect of the surgery that needs improving. Current methods of ureteroscopy take the physician only 15-20 minutes to break up the stone, but potentially another hour or more removing all of the stone fragments resulting from the lithotripsy. The physician must search through the urinary system and into the calyces of the kidney to locate these fragments, as their movement is completely sporadic.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

The embodiments of the present disclosure may provide an apparatus used in ureteroscopy, a widespread method of treating kidney stones by enclosing the stone in a sheath before breaking it into a plurality of fragments with a fragmenting device. The configuration of the disclosed mesh top to a sheath may improve the practice of ureteroscopy by facilitating the removal of kidney stone fragments. The apparatus may comprise a sheath; a mesh top comprising: a first end having an opening into the interior of the mesh top, and a second end being attached to the sheath; and an enclosing means, wherein the mesh top is configured to: receive an object within an interior of the mesh top, enclose, via the enclosing means, the object within the mesh top, receive a fragmenting device through a second end, and retain fragments of the object caused by an operation of the fragmenting device.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 illustrates an embodiment of an apparatus consistent with embodiments of the present disclosure;

FIGS. 2A-2B illustrate an embodiment of a handle consistent with embodiments of the present disclosure;

FIG. 3 is a flow chart of a method for providing an apparatus implementing a sheath for enclosure and removal of foreign bodies;

FIG. 4 illustrates the insertion of a guidewire in the direction of a kidney stone;

FIG. 5 illustrates the insertion of the apparatus over the guidewire;

FIG. 6 illustrates an inserted ureteroscope;

FIG. 7 illustrates a basket capturing a kidney stone;

FIG. 8 illustrates a kidney stone being pulled into the apparatus;

FIG. 9 further illustrates a kidney stone being pulled into the apparatus;

FIG. 10 illustrates a mesh top being closed around the stone;

FIG. 11A illustrates a captured kidney stone;

FIG. 11B illustrates a captured kidney stone being pulled into a mesh top;

FIG. 11C further illustrates a captured kidney stone being pulled into a mesh top;

FIG. 11D illustrates a mesh top being closed around the stone;

FIG. 12 illustrates stone fragmenting within a closed mesh top; and

FIG. 13 illustrates the removal of the smaller, more manageable pieces of kidney stone.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. As should be understood, any embodiment may incorporate only one or a plurality of the above-disclosed aspects of the display and may further incorporate only one or a plurality of the above-disclosed features. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. §112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present discloser includes many aspects and features. Moreover, while many aspects and features relate to, and are described in, the context of kidney stone removal, embodiments of the present disclosure are not limited to use only in this context.

### I. Apparatus Overview

Consistent with embodiments of the present disclosure, an apparatus for the removal of calculi and foreign bodies may be provided. This overview is provided to introduce a selection of concepts in a simplified form that are further described below. This overview is not intended to identify key features or essential features of the claimed subject matter. Nor is this overview intended to be used to limit the claimed subject matter's scope. The apparatus implementing a sheath for removal of foreign bodies may be used by individuals or companies to assist in the process of removing kidney stones.

The embodiments of the present disclosure may provide a cylindrical mesh top configurable to a sheath used in ureteroscopy. Although the disclosed mode of implementation of the mesh top is illustrated with reference to the capture of kidney stones, it should be understood that the embodiments disclosed herein may be adapted to accommodate the capture of calculi and foreign bodies (e.g., renal calculi or kidney stone fragments). Furthermore, although the disclosed mode of implementation is illustrated within a kidney, it should be understood that the embodiments disclosed herein may be used within the body (i.e. throughout the ureter or renal pelvis).

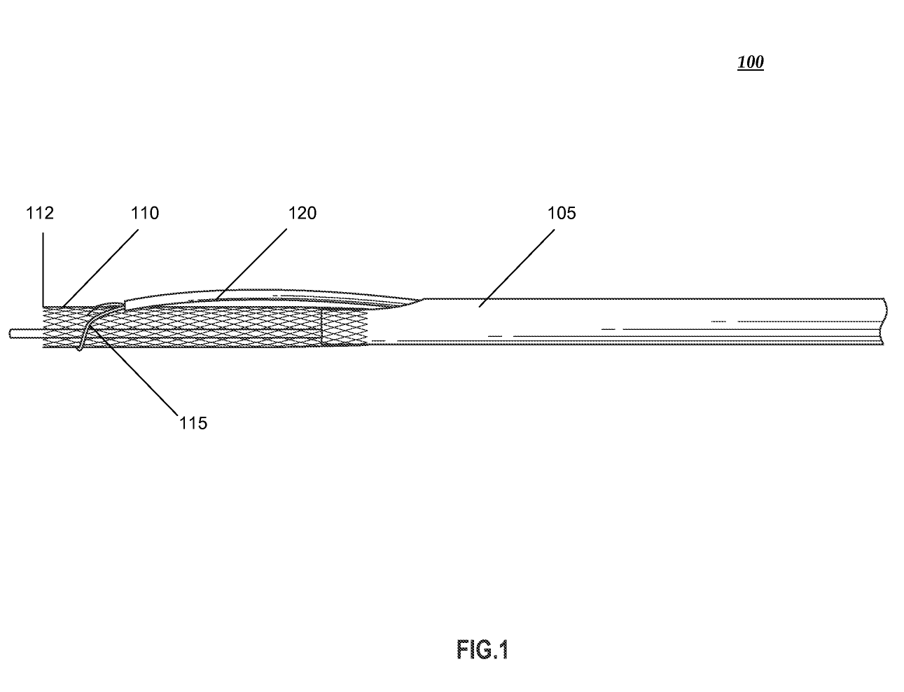

FIG. 1 illustrates an improved sheath and mesh top 100 of the present disclosure. The improved apparatus may comprise a mesh top 110 used in configurable conjunction with a sheath 105. Mesh top 110 may be configured with an enclosing means 115 at approximately an open end 112 of mesh top 110. In some embodiments, enclosing means 115 may comprise, for example, but not be limited to, a loop knot. Open end 112 may be employed to receive a foreign body into mesh top 110, while enclosing means 115 may be employed to enclose the foreign body captured within mesh top 110. Methods of receiving the foreign body into method top 105 will be detailed below.

Enclosing means 115 may be regulated by, for example, but not limited to, a tension cord operated through a channel 120 by a tension switch. Such regulation would enable a closing of open end 112 of mesh top 110. It should be understood that enclosing means 115, tension cord channel 120, and a tension switch may be replaced with any suitable contraction mechanism used to close open-end 112. In some embodiments, tension cord channel 120 may be used as a “backbone” to mesh top 110, providing structural stability throughout use in a ureteroscopy procedure.

During the ureteroscopy procedure, sheath 105 may be employed to encompass and pass a plurality of devices into the kidney including, but not limited to, a lithotripter. Although the embodiments disclosed herein reference a lithotripter, any suitable fragmenting device may be employed throughout the various embodiments of the present disclosure. Mesh top 110 consistent with embodiments of the present disclosure may be designed to accommodate various lithotripters configured to pass through sheath 105, including, for example, but not limited to, electrohydraulic, pneumatic, ultrasonic, or laser devices. Embodiments of the present disclosure may be employ such lithotripters to fragment a kidney stone once the kidney stone is received within mesh top 110 and open end 112 has been closed to secure the foreign body within mesh top 110.

Unlike conventional lithotripter applications, however, embodiments of the present disclosure may call upon the initiation of the fragmentation process once the stone is captured within enclosed mesh top 110. In this way, once fragmented, the fragments of the stone may remain within the enclosed mesh top 110 without escaping back into the kidney, thereby eliminating the process of individual fragment collection.

Still consistent with embodiments of the present disclosure, and as will be further detailed below, mesh top 110 may be comprised of a flexible, interwoven fabric (e.g., a braided closed-loop material) designed for expansion and contraction along an inner diameter of mesh top 110. For example, as a stone is received through open end 112, the force of the stone against mesh top 110 may cause a radial expansion of mesh top 110. The compressive force, in turn, may cause open end 112 to dilate (e.g., an increase in the mesh top's inner diameter). The expansion may facilitate the entry of a stone into mesh top 110.

Further still, as the resultant stone fragments are pulled out of the kidney (by removal of the sheath), the friction between the walls of the passage and mesh top 110 may create a tension, causing mesh top 110 to stretch and elongate. In turn, the extension causes mesh top 110 to more tightly enclose the fragments (due to the decreased inner diameter), thereby ensuring that fragments remain securely within mesh top 110. Consequently, the removal of a plurality of calculi fragments may be facilitated in one pass, reducing the time, discomfort, and risk of recurrence associated with treatment.

By enclosing the stone in mesh top 110 before the fragmentation process, embodiments of the present disclosure may eliminate the need to locate and remove resultant fragments, thereby shortening total procedure time substantially. Moreover, since the resultant fragments are readily captured within the enclosed mesh top 110 and subsequently, are removed, re-aggregation will not occur, reducing readmissions for that reason. Accordingly, patients and their families, hospitals, physicians, and payers will benefit from the use of the embodiments disclosed herein.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

### II. Apparatus Configuration

FIG. 1 illustrates an improved sheath and mesh top 100. It should be understood that a mesh top 110 and an improved sheath 105 comprising an integrated mesh top 110 may be individually, collectively, and interchangeably referred to as an “apparatus” throughout the embodiments of the present disclosure. Accordingly, FIG. 1 may be said to disclose an apparatus 100.

Apparatus 100 may comprise, but not be limited to, for example, a sheath 105, a mesh top 110, an enclosing means 115, and a tension cord channel 120. The sheath 105 may be comprised of a hydrophilic coating material, such as, for example, but not limited to a Fluorinated ethylene propylene (FEP)-coated outer sheath. The sheath 105 may provide a working channel for the procedure. A dilator (not shown) may enable deployment of apparatus 100 through a urethra of a subject (e.g., a patient with kidney stones). The dilator may be coated in hydrophilic material, such as, for example, but not limited to Polytetrafluoroethylene (PTFE).

A mesh top 110 may be implemented for capturing calculi or foreign body within the kidney of the subject (i.e., kidney stone). Mesh top 110 may be fabricated from a braided closed-loop material. The material may be comprised of for example, but not limited to, nitinol per ASTM F2063. An enclosing means 115, such as, for example, but not limited to, a loop knot tying mechanism, may be implemented towards an open end 112 of mesh top 110. In some embodiments, enclosing means 115 may be integrated within the fabric of mesh top 110 and positioned approximately towards open end 112. The closing means 115 may be comprised of, but not limited to, for example, a monofilament prolene line.

A channel for a monofilament prolene closure line 120 may run through sheath 105. The line tension may be controlled by a tension switch implemented at an end of sheath 105. In this way, increasing tension in the line may cause a tightening of the loop knot, thereby enclosing open end 112 of mesh top 110.

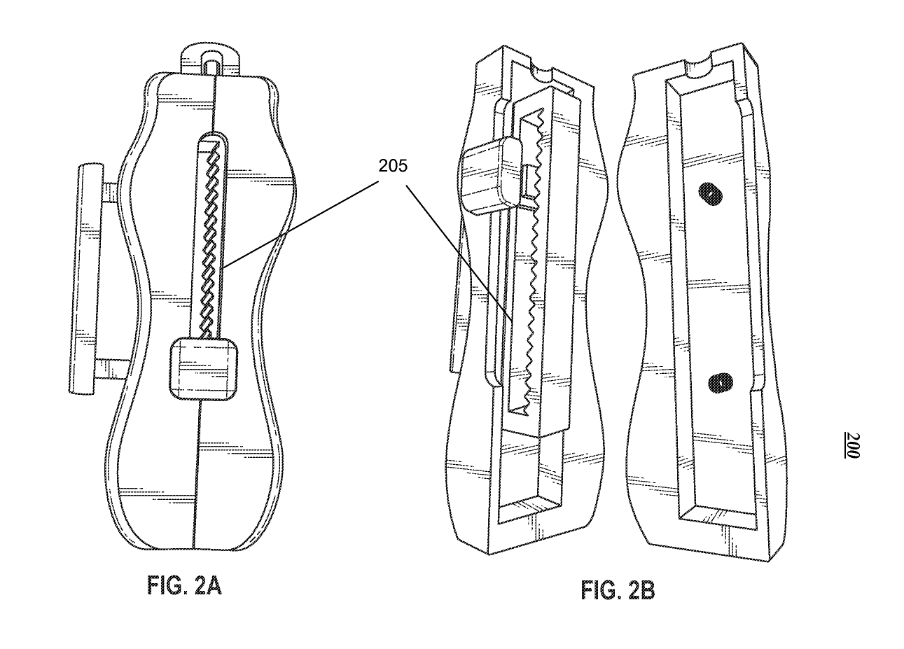

FIGS. 2A-2B illustrate a handle 200 consistent with embodiments of the present disclosure. Handle 200 may be implemented at the base of apparatus 100. The handle may be comprised of, but not limited to, plastic, such as, for example Acrylonitrile butadiene styrene (ABS). A tension switch, or ratcheting means 205 placed within handle may enable an operator to guide the apparatus as well as open and close the mesh top. Ratcheting means 205 may be spring-loaded and enable the operator to operate enclosing means 115.

It should be understood that enclosing means 115, tension cord channel 120, handle, and ratcheting means 205 may be replaced with any suitable contraction mechanism used to close open-end 112. In some embodiments, the material of sheath 105 may be extruded over the mesh top 110 to create junction. Still other techniques and methods may be employed in combing the sheath 105 with mesh top 110.

### III. Apparatus Operation

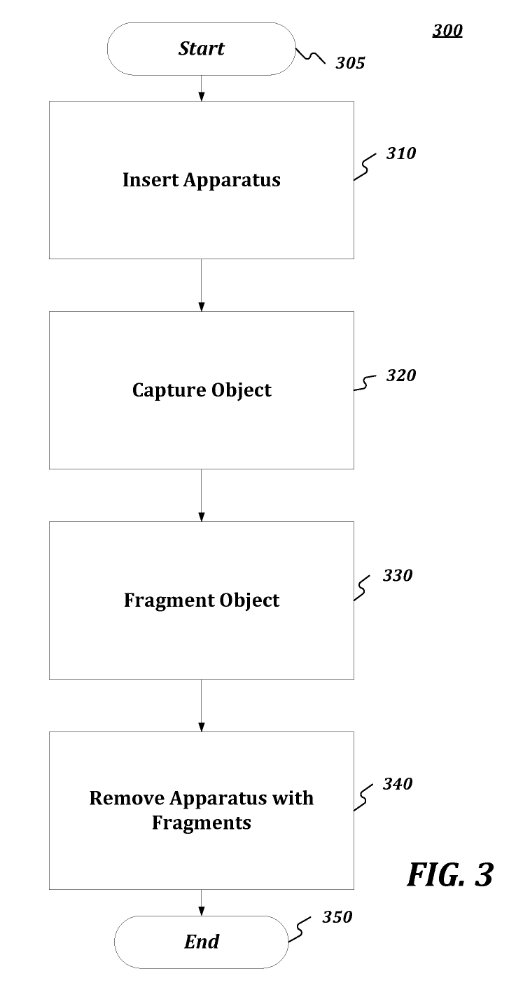

FIG. 3 is a flow chart setting forth the general stages involved in a method 300 consistent with an embodiment of the disclosure for operating the apparatus implementing a sheath for removal of foreign bodies.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 300 will be described in greater detail below. Further, although method 300 describes a method for kidney stone removal in a specific way, apparatus 100 may not be limited to such procedures.

Method 300 may begin at starting block 305 and proceed to stage 310 where apparatus 100 may be inserted into the kidney through the urethra. Prior to insertion, an insertion channel of the subject (e.g., urethra) may undergo dilation. Further still, a guide-wire may first be inserted into the channel (e.g., into the kidney through the urethra).

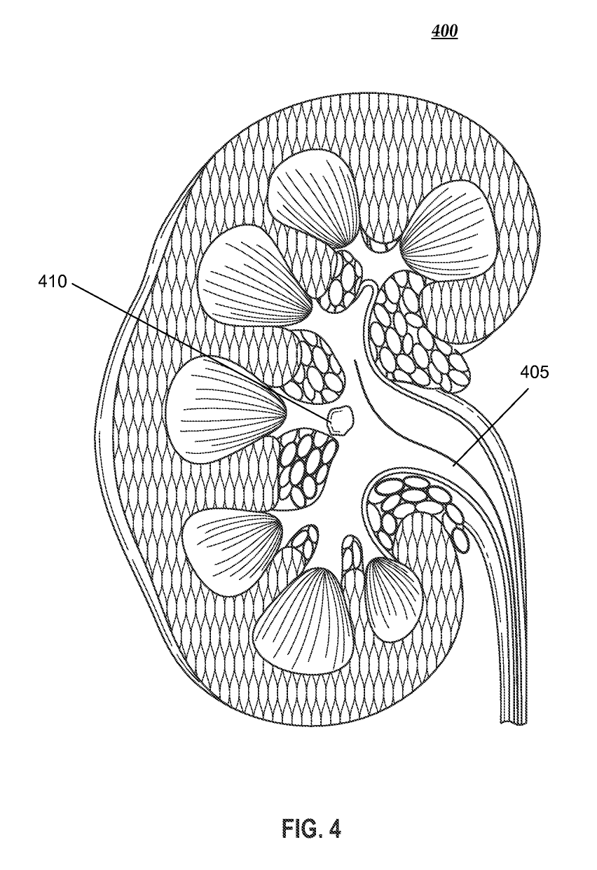

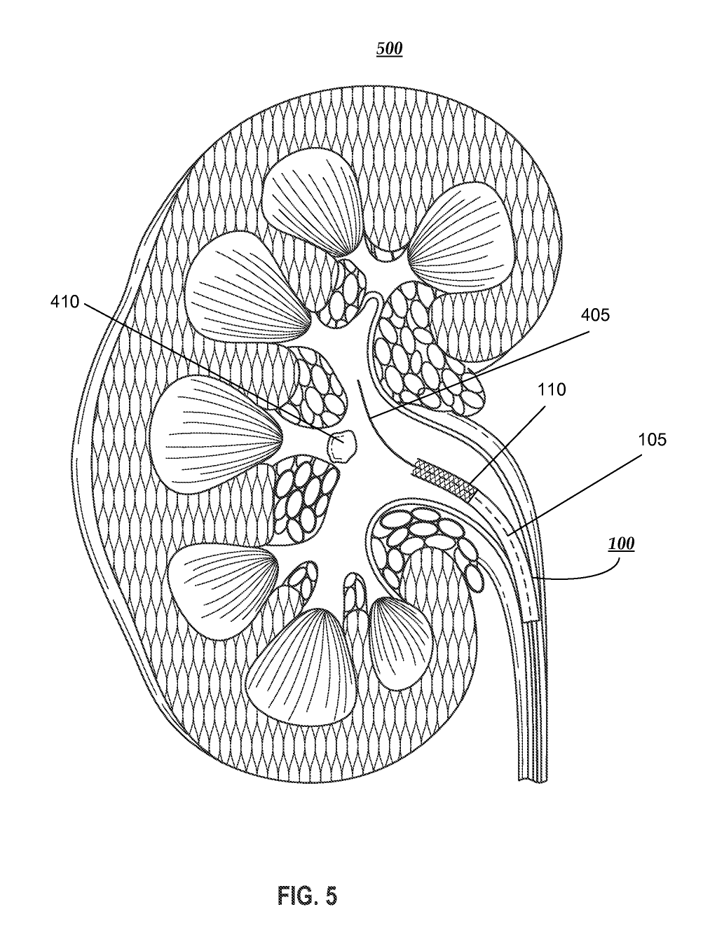

FIG. 4 illustrates the insertion of the guide-wire 405 in the direction of a target object 410 (e.g., calculi or foreign body). Then, apparatus 100 may be inserted over the guide-wire. FIG. 5 illustrates the insertion of the apparatus over the guide-wire. In some embodiments, a radiopaque ring may be used to make sure apparatus 100 is properly placed. Upon the insertion of apparatus 100 into the kidney, the guide-wire (and dilator) may then be removed.

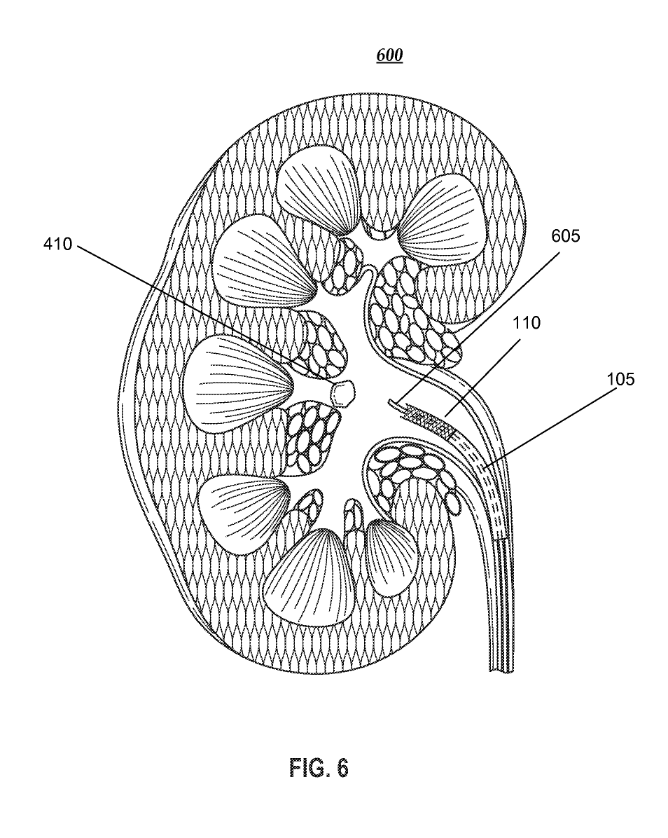

Next, a ureteroscope may be inserted through the sheath 105 of apparatus 100. FIG. 6 illustrates an inserted ureteroscope 605. Ureteroscope 605 may be used for viewing target object 410 and guiding apparatus 100 towards the stone.

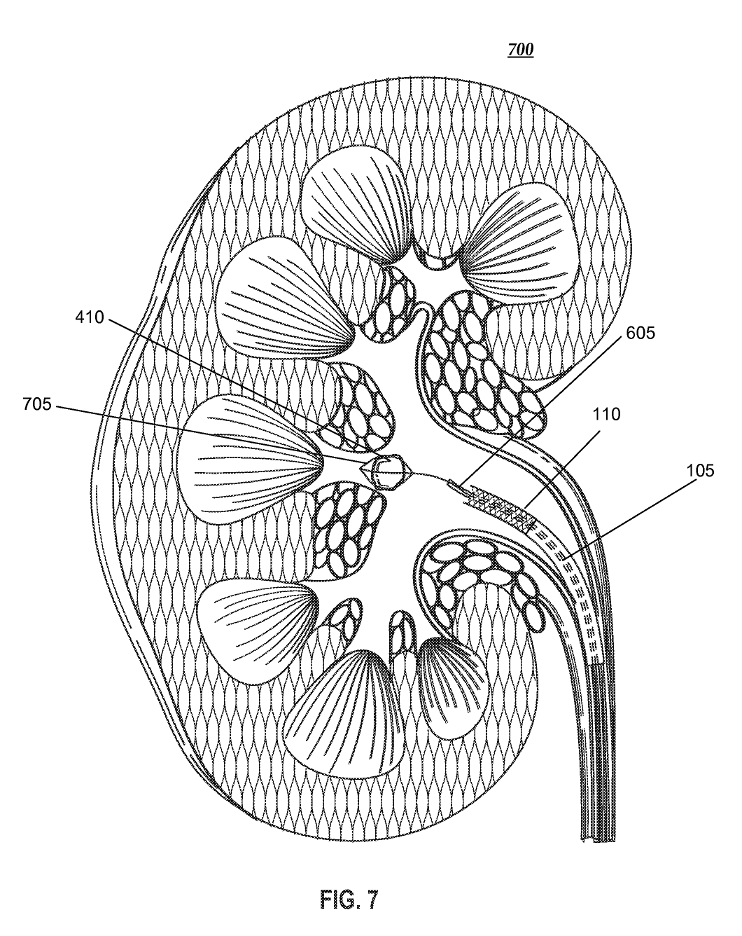

From stage 310, where apparatus 100 is inserted into the kidney, method 300 may advance to stage 320 where target object 410 may be captured within mesh top 110 of inserted apparatus 100. In some embodiments, capturing device (e.g., stone basket) may be employed to grasp and move target object 410 into mesh top 110. FIG. 7 illustrates such a capturing device 705 for capturing target object 410.

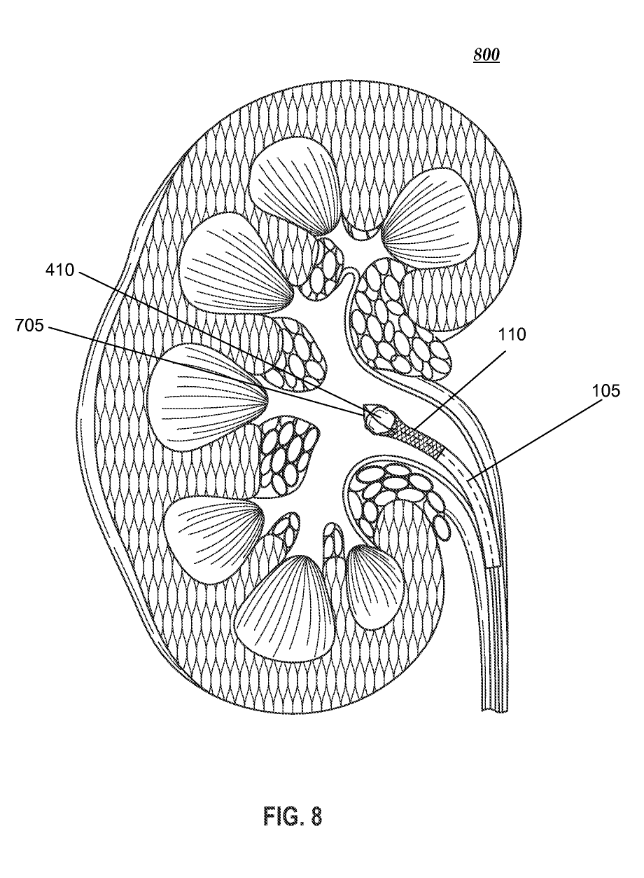

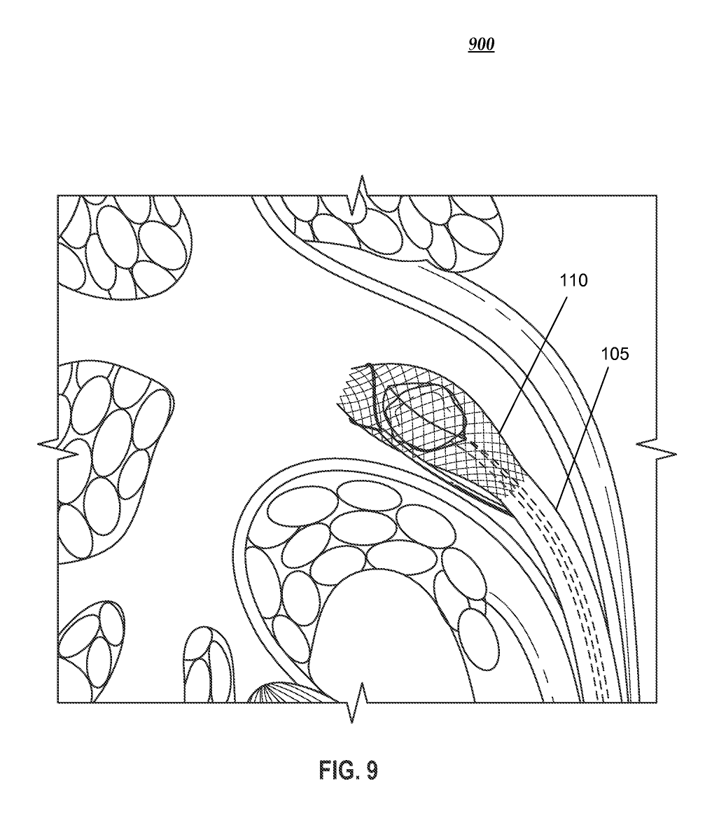

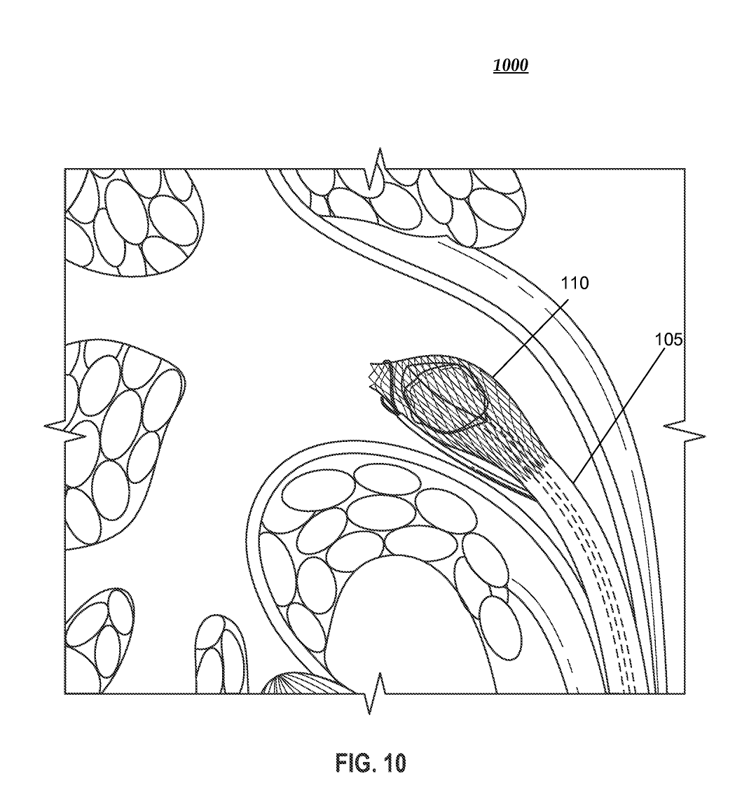

Capturing device 705 may be inserted into the kidney through apparatus 100. An operator may navigate capturing device 705 towards target object 410 and grasp target object 410. Capturing device 705 may be controlled by an operator and, upon grasping of object 410, may be pulled, along with object 410, into mesh top 110, as shown in FIG. 8 and FIG. 9. Once within mesh top 110, closing means 115 may be operated to secure target object 410 within mesh top 110. FIG. 10 illustrates a mesh top enclosing target object 410.

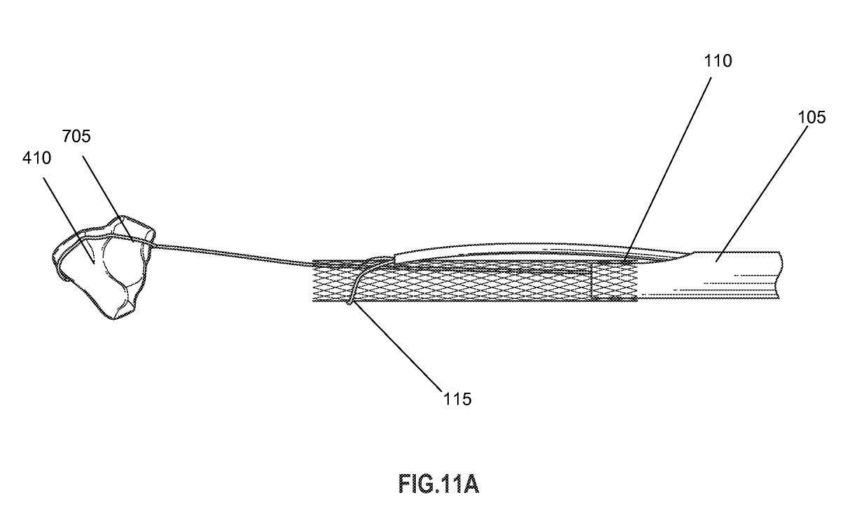

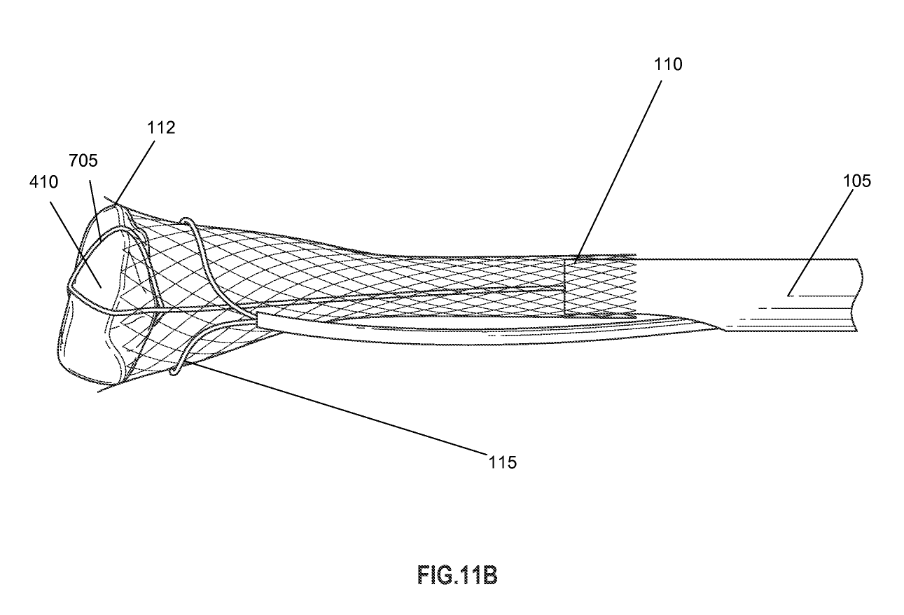

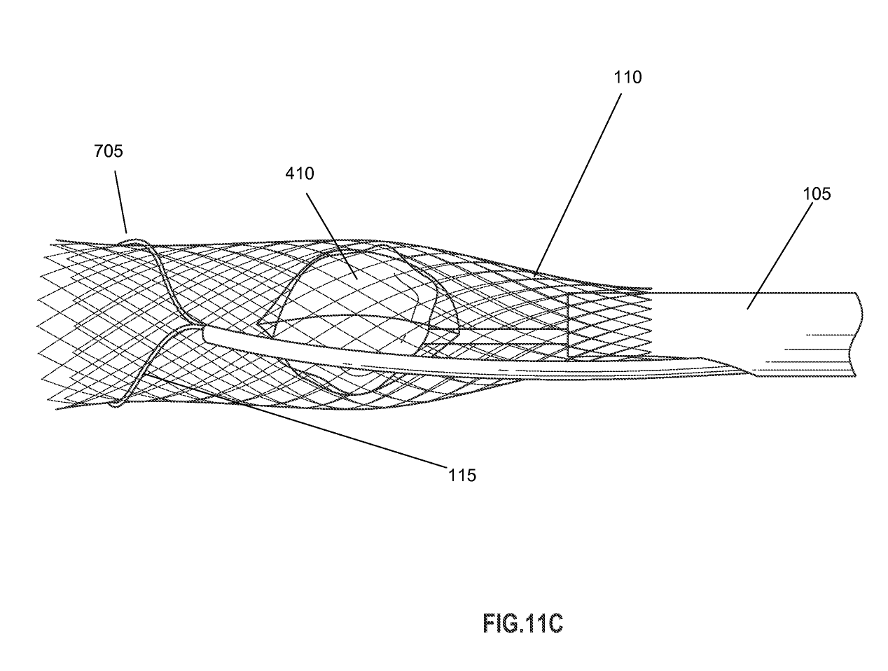

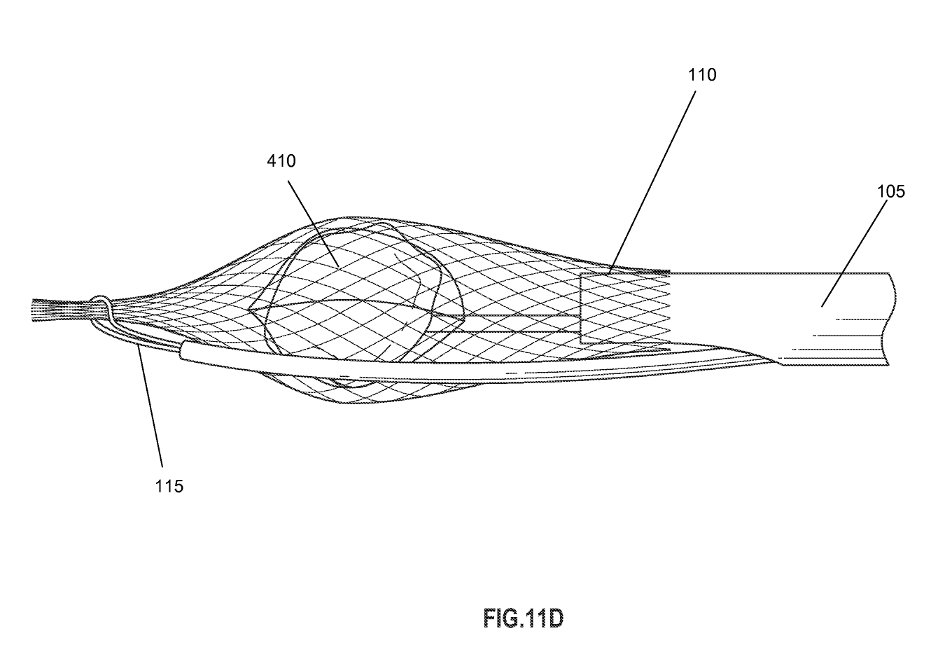

FIGS. 11A-D show another illustration of the capturing process. In FIG. 11A, target object 410 is grasped and captured by a capturing device 705 (e.g. basket), illustrated as tension line similar to that of enclosing means 115. The line may pull object 410 into mesh top 110. Upon receiving object 410 at open end 112 of mesh top 110, the resulting force from object 410 compresses mesh top 110 may cause mesh top 110 to expand the open end 112. In this way, the diameter of open end 112 may increase to accommodate the girth of object 410.

FIG. 11B shows the expansion as a result of the result of the closed braided fabrication of mesh top 110, designed to expand upon axial compression and stretch upon extension. In other embodiments of the present disclosure, the expansion of open end 112 may be regulated by enclosing means 115, which may be adapted to both enclose and enlarge open end 112. FIG. 11C illustrates the fabric of mesh top 110 stretching to secure and encompass object 410.

Referring now to FIG. 11D, enclosing means 115 may be operated to secure object 410 within mesh top 110 by closing open end 112. It should be understood that enclosing means 115 is illustrated conceptually. For example, various implementations of enclosing means 115 may call for the integration of the tension line within braided fabric of the mesh top 110. Still, other implementations may be provided so as to coincide with the utility of enclosing open end 112. The closure of open end 112 may also serve to more securely enclose object 410 within mesh top by the closing means 115.

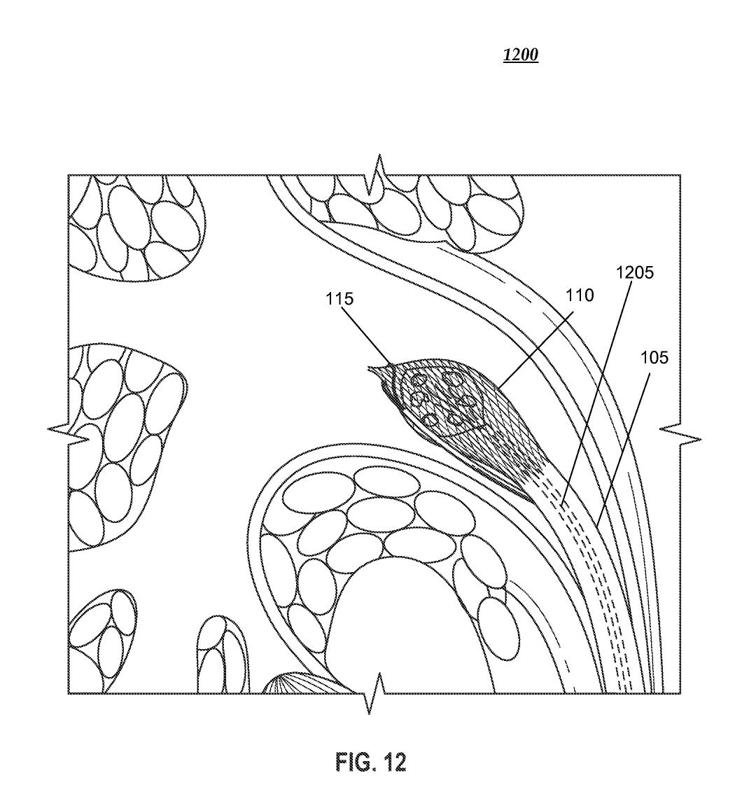

Once object 410 is captured in stage 320, method 300 may continue to stage 330 where object 410 may be physically fragmented. FIG. 12 illustrates an embodiment of object fragmentation. For example, a laser 1205 or other device (e.g., a lithotripter) for kidney stone ablation (e.g., electrohydraulic, pneumatic, ultrasonic devices) may be inserted through sheath 105 of apparatus 100. The lithotripter may protrude into mesh top 110 at a location approximately adjacent to object 410. In turn, fragmentation may be initiated within mesh top 110 or, in other words, in a controlled fragmentation zone.

Having fragmentation occur in a controlled fragmentation zone has plurality of advantages. For instance, the fragments may all be contained within mesh top 410, thereby eliminating the need to repeat the navigation and capture process for stone fragments. Additional advantages are listed below.

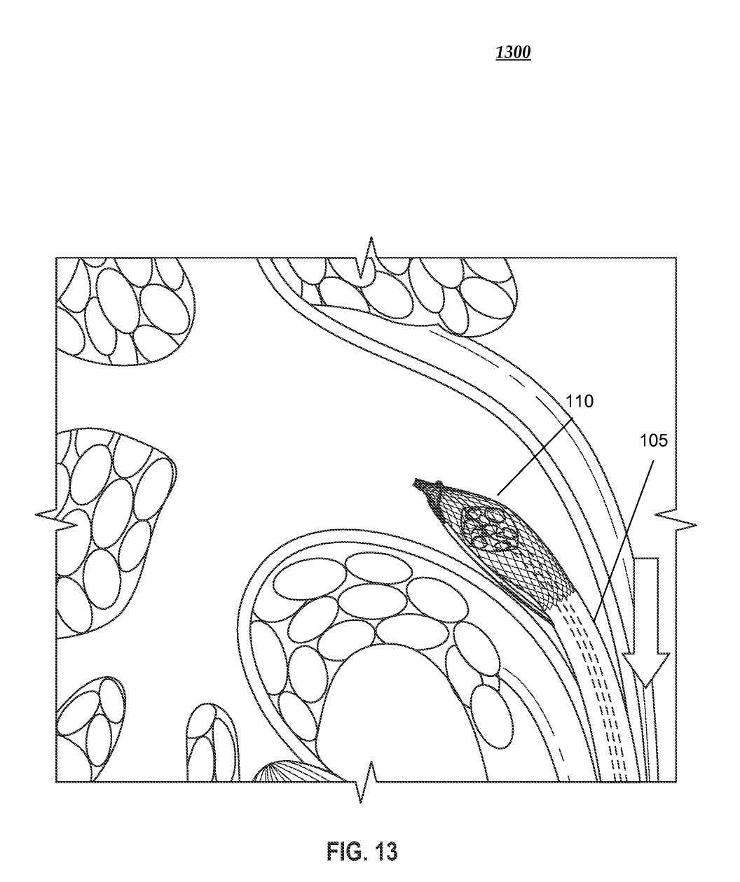

After object 410 is fragmented in stage 330, method 300 may proceed to stage 340 where the apparatus and stone pieces may be removed. Fragments, may more easily pass through the subject's channel (e.g., urethra) upon the withdrawal of apparatus 100 from the channel. Furthermore, withdrawal of apparatus 100 may create a friction between the subject's channel and mesh top 100. In some embodiments of the present disclosure, the friction may cause an extension of mesh top 110 which, in turn, due to mesh top 100's braided fabric design, cause a tightening (or decrease of inner diameter) of mesh top 100's grasp of object fragments. FIG. 13 illustrates the removal of the smaller, more manageable pieces of object 410. Once the apparatus and object fragments are removed in stage 340, method 300 may then end at stage 350.

### IV. Advantages

Apparatus 100 is may be easily incorporated into current procedures. For example, the stages illustrated by method 300 (some of which are undertaken to currently perform a ureteroscopy procedure) may remain substantially the same (with exception to, at least, the enclosure of object 410 within mesh top 110 prior to fragmentation. In this way, apparatus 100 may make a large impact with very little disturbance to the performance of conventional ureteroscopy procedures.

Additional advances of the various embodiments disclosed herein include, but are not limited to, for example:
Apparatus 100 may be embodied in a disposable design,
Apparatus 100 may not be dependent on basket, scope or lithotripter type, although it may be compatible with a variety of device types,
Does not require a kidney wall to trap the stone (stone may be trapped within mesh top 110),
Mesh top 110 may be attached directly to/integrated with sheath 105.

This is more practical because a practicing physician (e.g., apparatus 100's user) may not face a large learning curve as they may simply replace their existing sheath's with apparatus 100,
Since mesh top 110 encloses stones prior to fragmentation, a sweeping through the kidney may not be needed to recover stone fragments,
Apparatus 100 may work throughout the urinary system and not limited to the kidney, and
Apparatus 100 does not require ablation against the kidney walls.

### V. Claims

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

Although very narrow claims are presented herein, it should be recognized the scope of this disclosure is much broader than presented by the claims. It is intended that broader claims will be submitted in an application that claims the benefit of priority from this application.

The following is claimed:

1. A apparatus comprising:
a sheath;
a mesh top comprising:
a first end having an opening to an interior of the mesh top, and
a second end being attached to the sheath; and
an enclosing means,
wherein the mesh top is configured to:
receive an object within an interior of the mesh top,
enclose, via the enclosing means, the object within the mesh top,
receive a fragmenting device through a second end, and
retain fragments of the object caused by an operation of the fragmenting device.

2. The apparatus of claim 1, wherein the mesh top is comprised of a closed-loop braided material.

3. The apparatus of claim 1, wherein a diameter of the first end of the mesh top is configured to expand upon an axial compression of the mesh top.

4. The apparatus of claim 3, wherein the diameter expansion of the first end enables a passage of the object into the interior of the mesh top.

5. The apparatus of claim 1, wherein a diameter of the first end is configured to contract upon an extension of the mesh top.

6. The apparatus of claim 5, wherein the extension of the mesh top is configured to occur upon friction resulting from a propagation of the apparatus within a channel.

7. The apparatus of claim 1, wherein the enclosing means is integrated into the mesh top approximately near the first end of the mesh top.

8. The apparatus of claim 1, wherein the enclosing means is configured to substantially close the opening at the first end of the mesh top.

9. The apparatus of claim 1, wherein the mesh top is comprised of nickel titanium alloy.

10. An apparatus comprising:
a cylindrical closed-looped braided material;
a first end having an opening into an interior of the cylindrical material; and
a second end being configurable to a sheath,
wherein a diameter of the opening is configured to expand upon an application of a first axial compressive force at the first end, at a first vector pointing towards the second end, and
wherein the interior of the cylindrical material is configured to compress upon an application of a second axial force received at the second end, at a second vector pointing away from the first end.

11. The apparatus of claim 11, wherein the interior of the cylindrical material is configured to receive an object through the first end.

12. The apparatus of claim 12, further comprising an enclosing means integrated into the cylindrical material at a circumference approximately adjacent the first end.

13. The apparatus of claim 12, wherein the enclosing means comprises a loop knot tie.

14. The apparatus of claim 13, wherein the loop knot tight is operated by a tension line such that an increase in tension causes a closing of the first end.

15. The apparatus of claim 11, wherein the interior of the cylindrical material is configured to contain an object.

16. The apparatus of claim 16, wherein the second end is configured to receive a fragmenting device for breaking up the object into a plurality of fragments.

17. The apparatus of claim 16, wherein the fragmenting device is configured to be received at the second end through the sheath.

18. The apparatus of claim 16, wherein the interior of the cylindrical material is configured to contain the plurality of fragments.

19. A apparatus comprising:
a sheath;
a cylindrical mesh top comprised of a closed-loop braided material, the mesh top having:
a first end having an opening to an interior of the mesh top, and
a second end being attached to the sheath; and
an enclosing means integrated into a circumference of the mesh top, positioned at approximately the first end, the enclosing means comprising a tension line which, upon an increase of tension, encloses the opening of the first end;
wherein the mesh top is configured to:
expand upon a axial compression of the mesh top due to a force applied from an object at the first end, wherein the expansion causes an increase of a diameter of the first end, enabling a passage of the object into the interior of the mesh top,
enclose, via the enclosing means, the object within the mesh top,
receive a fragmenting device through a second end, and
retain fragments of the object caused by an operation of the fragmenting device.
